(19) United States
(12) Patent Application Publication
(10) Pub. No.: US 2012/0237647 A1
(43) Pub. Date: Sep. 20, 2012

(54) MULTI-FUNCTIONAL PLATFORM FOOD PREPARATION DEVICE

(76) Inventors: Marvin Dobert, Bolton Landing, NY (US); Mark Groenhuyzen, Huntington Belt, CA (US); Joseph Klingl, Los Angeles, CA (US); Jerry Sharber, Cypress, CA (US); Jack Lovley, II, Lake Forest, CA (US)

(21) Appl. No.: 13/223,277

(22) Filed: Aug. 31, 2011

Related U.S. Application Data
(63) Continuation-in-part of application No. 13/065,235, filed on Mar. 17, 2011, now abandoned.

(51) Int. Cl. A47J 37/07; G01N 33/02; A23L 1/01

(52) U.S. Cl. 426/243; 99/340; 426/438; 426/416; 426/478; 426/510; 426/509; 426/231

(57) ABSTRACT

A multi-functional platform food preparation device is disclosed which has the ability to fry, deep-fry, steam, stir fry, stage, drain, warm and grill foods conveniently on the same cooking platform. The cooking platform can include a built in reservoir for cooking soups, steaming with water, stir frying, and deep frying with oil. The cooking platform also can include a grilling section, a griddle/frying/cooking section, and a warming/draining section. The cooking platform also can include a drain chute connecting the reservoir to a catch bottle to aid in the clean up process. In addition, the reservoir can include a slosh ring positioned around the upper perimeter of the reservoir. Deep frying/boiling/steaming/stir frying, grilling, griddle/frying/cooking, and warming/draining sections can be contained on a single cooking platform to aid in the synchronized preparation of common meals to be prepared together in a fast, convenient and safe manner.

### RELATED APPLICATIONS

This application is a continuation-in-part of U.S. patent application Ser. No. 13/065,235, filed Mar. 17, 2011, the entire contents of which are incorporated herein by reference.

### BACKGROUND

1. Field of the Invention

The invention disclosed herein relates generally to the field of outdoor food preparation devices. More specifically, the invention relates to an outdoor food preparation device that has the capabilities of grilling food, deep frying/steaming/boiling/stir frying food, cooking/frying food by griddle, and warming/draining food.

2. Description of the Related Art

In recent years, popularity has grown with regard to the method of preparing foods by grilling, deep-frying/steaming/boiling/stir frying, cooking/frying, and warming food in outdoor settings with outdoor cooking appliances. While grilling, deep-frying/steaming/boiling/stir frying, cooking/frying, and warming food have always been popular methods of preparing and enjoying food, it is only in recent years that these methods have gained in great popularity with the general public. While many grills and fryers are usable to prepare food by a particular cooking method, they fail to combine the versatility and plurality of cooking methods in a single food preparation device.

The grilling, deep frying, stir frying, and frying/steaming process, while providing a method of preparing flavorful meals, traditionally requires several food preparation devices, auxiliary cooking parts, and many kitchen tools. As a result, grilling, deep frying, stir frying, and frying/steaming foods tend to be time and labor intensive. Although there are many food preparation devices that perform any one of the grilling, deep-frying/steaming/boiling/stir frying, cooking/frying, and warming foods, there are no outdoor food preparation devices that combine each of these cooking methods in a single food preparation device.

Known designs fail to put the emphasis of cooking on using several distinctly different methods of cooking to produce very common and popular meals that require grilling, deep-frying/steaming/boiling/stir frying, cooking/frying, and warming foods together.

Outdoor deep fryers and outdoor grills generally require the use of several different food preparation devices to accomplish the intended goal of cooking, grilling, deep-frying/steaming/boiling/stir frying, cooking/frying, and warming foods together in a safe manner. These designs require grilling on one device and providing other devices for frying, steaming, boiling, and warming foods. Generally, the main function, emphasis and performance for outdoor cookers is on the individual grilling or individually frying of foods. In order to grill food, deep fry/steam/boil/stir fry food, cook/fry food by griddle, and warm/drain foods equally and simultaneously requires additional devices, parts, components and utensils not specifically set up for the grilling or the frying methods of cooking. Preparing food with these different techniques requires a large amount of space and can be arduous and labor intensive. The process of including a reservoir for boiling/cooking/deep-frying/stir frying, frying/cooking and warming/draining with the traditional outdoor grill and all their accessories are often times cumbersome and difficult to transport inside and outdoors. Prior devices simply fail to provide for the smooth preparation and method of grilling, deep-frying/steaming/boiling/stir frying, cooking/frying, and warming foods simultaneously to facilitate preparation of some of the most popular meals.

### SUMMARY

A multi-functional platform food preparation device can be assembled, transported, cleaned, and stored easily.

In some embodiments, a multi-functional platform food preparation device comprises a cooking platform that comprises a first section that includes a reservoir for deep frying, steaming, stir frying, and boiling; a second section for grilling; a third section for cooking and frying by griddle; and a fourth section for warming and draining.

In some embodiments, a method of cooking comprises deep frying, steaming, stir frying, or boiling a first food; grilling a second food; cooking or frying by griddle a third food; and warming or draining a fourth food; wherein the deep frying, steaming, stir frying, or boiling of a first food, grilling of a second food; cooking or frying by griddle of a third food; and warming or draining of a fourth food occurs on a single cooking platform.

In some embodiments, a multi-functional platform food preparation device comprises a first pair of leg assemblies, wherein the first pair of leg assemblies includes a first pair of mounting brackets and a first pair of legs; a second pair of leg assemblies, wherein the second pair of leg assemblies includes a second pair of mounting brackets and a second pair of legs; wherein the first pair of mounting brackets are larger than the second pair of mounting brackets; and wherein the second pair of legs are longer than the first pair of legs.

### BRIEF DESCRIPTION OF THE DRAWINGS

FIG. 1 is an exploded front perspective view of a food preparation device.

FIG. 2A is a perspective view of a cooking platform.

FIG. 2B is a top view of a cooking platform.

FIG. 2C is a side view of a cooking platform.

FIG. 3 is an enlarged perspective view of the warming/draining component of a cooking platform.

FIG. 4A is a top perspective view of a food preparation device.

FIG. 4B is a front view a food preparation device.

FIG. 4C is a rear view of a food preparation device.

FIG. 4D is a bottom perspective view of a food preparation device.

FIG. 5 is a perspective view of a slosh ring and thermometer.

FIG. 6A is a partial side view of a food preparation device showing the cooking platform in a horizontal position.

FIG. 6B is a partial side view of a food preparation device showing the cooking platform in a raised position.

FIG. 7 is a top view of a food preparation device with the cooking platform removed to show the heating elements.

FIG. 8 is a bottom perspective view of selected portions of the food preparation device.

FIG. 9A is a side view of open legs of a food preparation device in a rotated position.

FIG. 9B is a side view of open legs of a food preparation device showing an offset between legs.

FIG. 9C is a top view of open legs of a food preparation device.

FIG. 9D is a bottom view of closed legs of a food preparation device.

### DETAILED DESCRIPTION OF THE PREFERRED EMBODIMENTS

A food preparation device with the plural combination of grilling food, deep frying/steaming/boiling/stir frying/fonduing food, cooking/sauteing/frying food by griddle, and warming/draining food can all be contained on one cooking platform. A built-in reservoir can be integrated into a cooking platform to provide the capability for deep-frying, stir frying, heating soups, and boiling and steaming foods. Also, an open flame grilling component, frying/griddle/cooking component, and warming/draining component can be integrated into the food preparation device on the same cooking platform as the built-in reservoir. Although each of these components can be of equal size in relation to the other components, it will be appreciated that each component can range in size and shape from small to large depending on the specific cooking needs of a user. In addition, although the cooking platform can be one continuous piece of material, lending to ease and simplicity of manufacturing, cooking and cleaning, it will be appreciated that it also can be made in separate components. The operator of the food preparation device can synchronize the preparation of meals in a simple, safe and manageable fashion.

The food preparation device can be simple and easy to assemble, maintain, transport, clean, and store.

Another feature of the food preparation device is its ability to recapture liquid from food placed in the warming/draining component to the reservoir. The warming/draining component of the food preparation device is designed with an inclined pitch to allow excess oil and/or water to drain off of food and be recaptured back into the reservoir. To prevent food placed on the warming/draining component from sliding back into the reservoir, the warming/draining component can include one or more blocking knobs near the mouth of the warming/draining component.

The reservoir on the cooking platform for cooking/steaming/boiling/stir frying and deep-frying can be located in the center of the cooking platform, but it also may be located anywhere on the cooking platform depending on the specific cooking needs. Although the reservoir can be of a traditional bowl shape, the reservoir may vary in shape, size, and volume depending on the overall size of the cooking platform and specific cooking needs. As the overall size of the cooking platform increases, the size of the reservoir can increase as well. Alternatively, the size of the reservoir could remain the same as the size of the cooking platform increases.

In preferred embodiments, the cooking platform can be circular in shape. The overall diameter of the food preparation device platform can vary in size depending on cooking requirements. In addition, the overall shape of the food preparation device may be square, rectangular, triangular, or any other shape, and have different configurations of the four cooking components depending on the specific cooking requirements.

The grilling component of the food preparation device can have the same inclined pitch or be more level for grilling. The grilling surface can have openings in the surface to enable the food to be in direct contact with the heating element, enabling the grilling of foods over an open fire. The grilling heating element can be electric or gas. In preferred embodiments, the grilling heating element comprises an infrared burner.

The griddle/frying component of the food preparation device is generally relatively flat to accommodate independent cooking/frying/griddling that can be used to cook eggs, pancakes, vegetables, meats and other items. In other embodiments, the griddle/frying component can be of any size, shape and tilt angle to accommodate the specific cooking requirements of the user.

In preferred embodiments, the cooking platform can be a unitary metal structure. However, it will be appreciated that the overall construction of the food preparation device can be made in separate parts or sections to accomplish the same purpose. Although the device can be made from stamped and shaped aluminum and steel, the device also may be shaped, stamped, spun, forged or made by other means from metal, ceramic or combination thereof, or any other usable cooking material of sufficient thickness to support the weight of the food item being cooked.

The cooking platform can be removable from the lower housing to allow for cleaning and for the interchangeability and use of other cooking platforms.

The heat source of the food preparation device can be fueled by liquid petroleum gas, including propane. In addition, natural gas, charcoal, gelled petroleum, electricity, green gas, or any other fuel also can be used. Furthermore, any combination of liquid petroleum gas, propane, natural gas, charcoal, gelled petroleum, electricity, and green gas can be used to provide energy for the heating elements. In preferred embodiments, the burners are independent for each cooking component. In other embodiments, the cooking sections can share burners.

The level of heat for the grilling heating element, frying/griddle heating element, and the deep frying/boiling heating element can be operated by separate controls. Alternatively, the level of heat for the heating elements can be controlled by a single control depending on the configuration.

A cover or lid to enclose the cooking platform can be of benefit when attempting to maintain the moistness of the food being grilled or fried/steamed. The food preparation device can include a lid that can be used to interchangeably cover any of the grilling component, frying/griddle/cooking component, and warming/draining component.

The food preparation device preferably can assemble and disassemble quickly for efficient outdoor and backyard use. To ease assembly and disassembly, the food preparation device can include a collapsible stand. When the collapsible stand is in a folded position, the food preparation device takes up very little space and is easy to transport. A carry bag can be provided to further facilitate ease of transport. In alternative embodiments, the food preparation device could be permanently installed in particular locations. In such embodiments, the food preparation device could be modified accordingly to accommodate any permanent location.

The food preparation device disclosed herein also facilitates food preparation in a group environment. For example, in preferred embodiments, the cooking platform is circular in shape, the deep frying/steaming/stir frying reservoir can be located at the center, and the grilling section, griddle/cooking/frying section, and warming/draining section 240 can be located on the periphery of the cooking platform. This arrangement of cooking surfaces allows several people to easily be simultaneously situated around the food preparation device and participate in the cooking process. As such, the food preparation device can enhance preparing food for groups of people, such as when tailgating. It should be appreciated that this benefit is not limited to embodiments in which the cooking platform is circular. For example, embodiments in which the cooking platform is shaped differently or in which the cooking sections are placed in different locations on the cooking platform can all allow a group of people to be situated around the food preparation device and interact with the cooking process.

Often times after frying in oil, the general public is confronted with the dilemma of what to do with the used or old cooking oil. The disposal of the oil often times is not anticipated during the preparation of frying food and ends up getting spilled or dumped in less than desirable places in the process of disposal.

The food preparation device can provide an easy and convenient way to dispose of used or old oil. When the oil has cooled to a temperature that is adequate for handling without risk of burning a person, the food preparation device provides for oil or water to be drained into a container in a controlled manner. In particular, the cooking platform can have a grooved drain channel in the flange that naturally routes the liquid to a mouth located on the outer edge of the cooking platform. The mouth can act as a funnel that allows for easy transfer of liquid to most common containers. The draining mechanism on the food preparation device creates an easy way to controllably pour the liquid from the drain groove to the mouth of a catch bottle.

For the purpose of understanding particular embodiments, reference will now be made to the drawings.

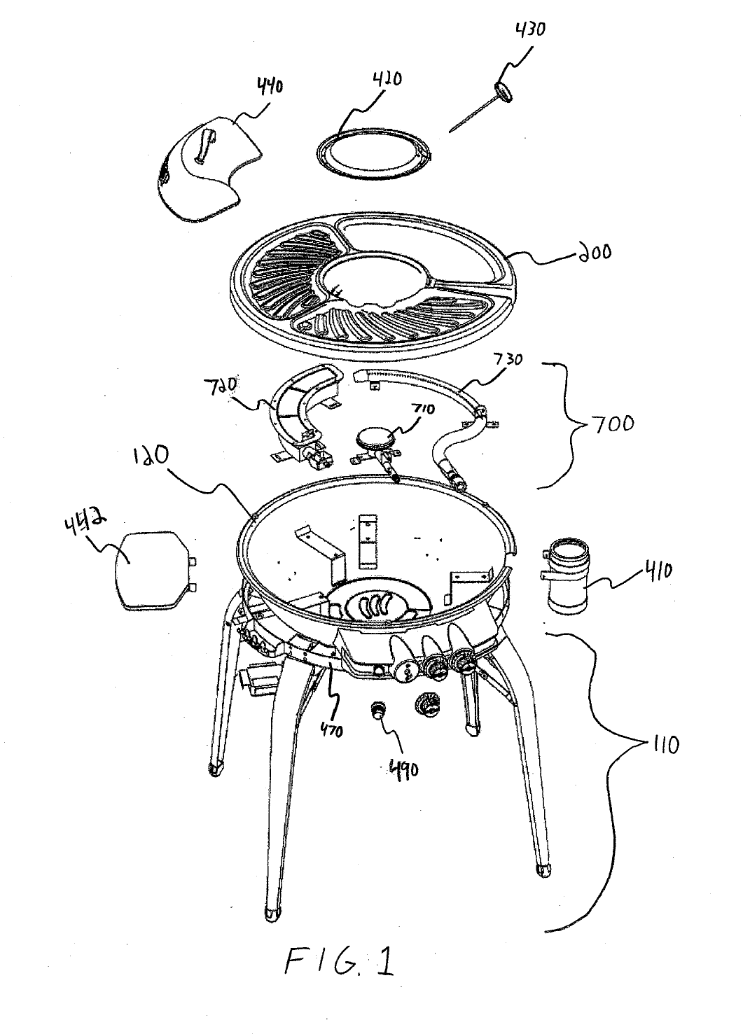

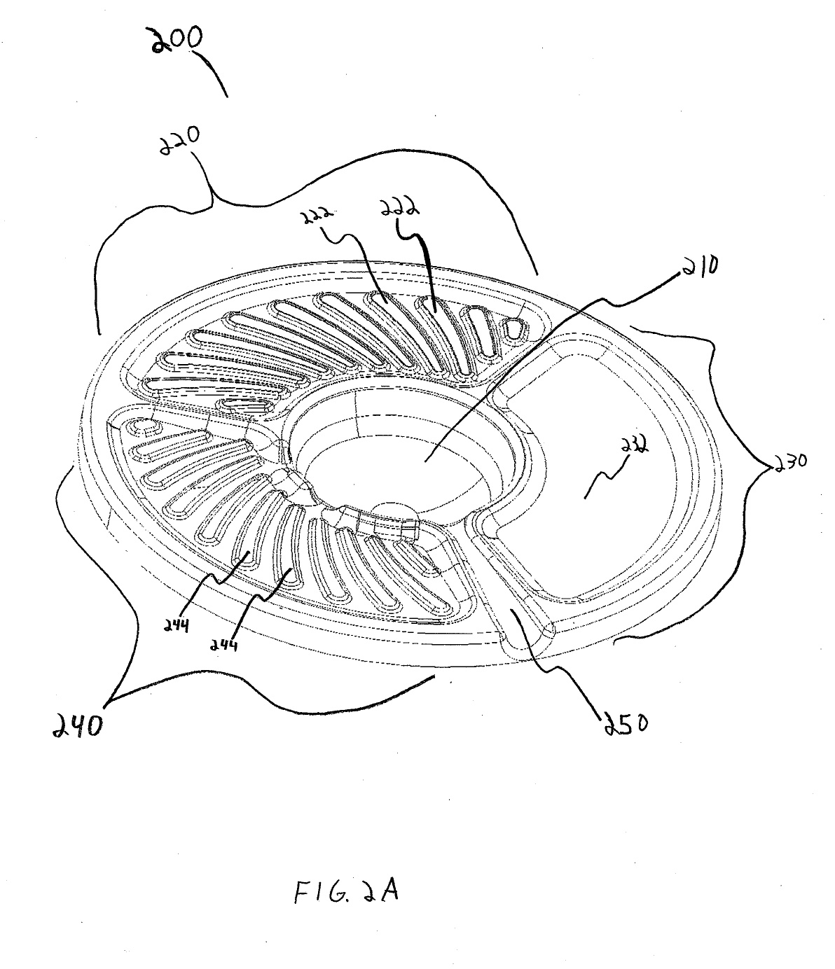

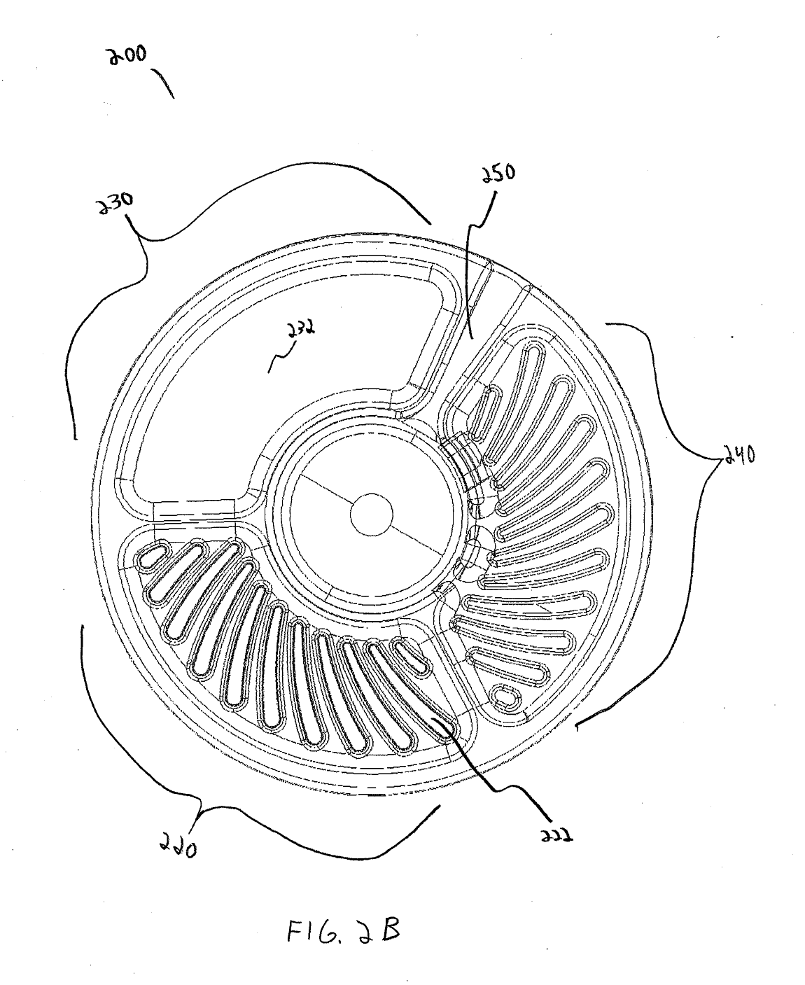

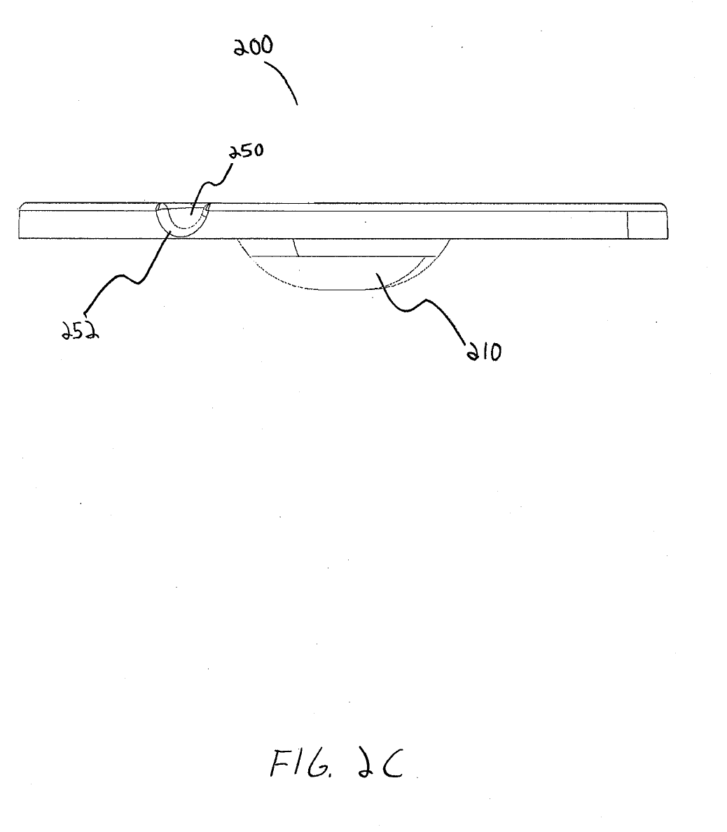

FIG. 1 illustrates an exploded front perspective view of some embodiments of a multi-functional platform food preparation device. In particular, the food preparation device of FIG. 1 includes, among other things, a stand 110, a lower housing 120, a cooking platform 200, a catch bottle 410, a slosh ring 420, a thermometer 430, a removable lid 440, a side table 442, a frame ring rail 470, an igniter 490, and heating elements 700, which includes fryer/steamer burner 710, an infrared grill burner 720, and griddle burner 730. The side table 442 can attach to the frame ring rail 470 of the stand 110. These components will be described in more detail in reference to FIGS. 2-9.

FIGS. 2A-2D illustrate varying views of a cooking platform 200 included in some embodiments of a multi-functional platform food preparation device. In particular, FIG. 2A illustrates a perspective view of the cooking platform 200, FIG. 2B illustrates a top view of the cooking platform 200, and FIG. 2C illustrates a side view of the cooking platform 200. In preferred embodiments, the cooking platform 200 includes a deep frying/steaming/stir frying reservoir 210, a grilling section 220, a griddle/cooking/frying section 230, a warming/draining section 240, and a drain chute 250. The cooking platform 200 preferably can be formed as a single unitary structure in a circular shape. Alternatively, the cooking platform 200 can be formed from component pieces and can be any other shape. For example, the cooking platform 200 can be rectangular or pentagonal, and additional cooking sections can be added or removed to accommodate the particular shape used for the cooking platform. In some embodiments, the cooking platform 200 can be coated with a porcelain cooking surface, thereby making it easier to clean.

In alternative embodiments, additional cooking sections can be added to the cooking platform 200. For example, four, five, six, or more separate cooking sections can be to the cooking platform. The only limit to the number of cooking sections is the size of the cooking platform. In some alternative embodiments, an additional reservoir can be included. For example, the additional reservoir could be used for boiling foods, whereas the other reservoir could be used for frying foods.

A reservoir 210 for deep frying, steaming, stir frying, and boiling can be included in the center of the cooking platform 200. The reservoir 210 can be of varying shapes and sizes depending on the desired capacity. The reservoir 210 preferably should be of sufficient size to accommodate oil used for deep frying or stir frying and water used for boiling or steaming food. In some embodiments, the reservoir 210 has a capacity of 16 oz. As shown in FIG. 2C, the reservoir 210 can extend down relative to the surrounding area of the cooking platform. In alternative embodiments, the reservoir 210 may be located at other positions of the cooking platform.

The periphery of the cooking platform 200 can include a grilling section 220, a griddle/cooking/frying section 230, and a warming/draining section 240. The grilling section 220 may be used to provide a method of cooking in which there is direct contact to an open flame through a plurality of open slots 222. The plurality of open slots 222 allows direct radiation and heat to penetrate food such as burgers, steaks, vegetables and such. Although the plurality of open slots 222 is illustrated as curvilinear in shape, any shape of open slots may be used.

The griddle/cooking/frying section 230 can include a continuous surface 232 without any direct openings to the open flame or heat source. The continuous surface 232 preferably can be substantially flat but also can be curved or titled. The griddle/cooking/frying section 230 can cook foods that cannot be grilled such as eggs, pancakes, and any foods in which it is desirable to retain juices or sauces for consumption. The griddle/cooking/frying section 230 compliments the other sections on the cooking platform 200.

The warming/draining section 240 fully compliments the other sections of the cooking platform. For example, the warming/draining section 240 can be used as a staging area for food recently cooked and ready for consumption. Elevated grooves 244 can support the food while oil, water, or other sauces drain back into the reservoir 210.

The cooking platform 200 can also include a drain chute 250 that extends from the reservoir 210 to the outer edge of the cooking platform 200. As shown in FIG. 2C, the drain chute 250 can include a mouth 252 at the outer edge of the cooking platform 200. The drain chute 250 provides for the easy and controlled cleanup of used oil, water or other viscous material for disposal or reuse.

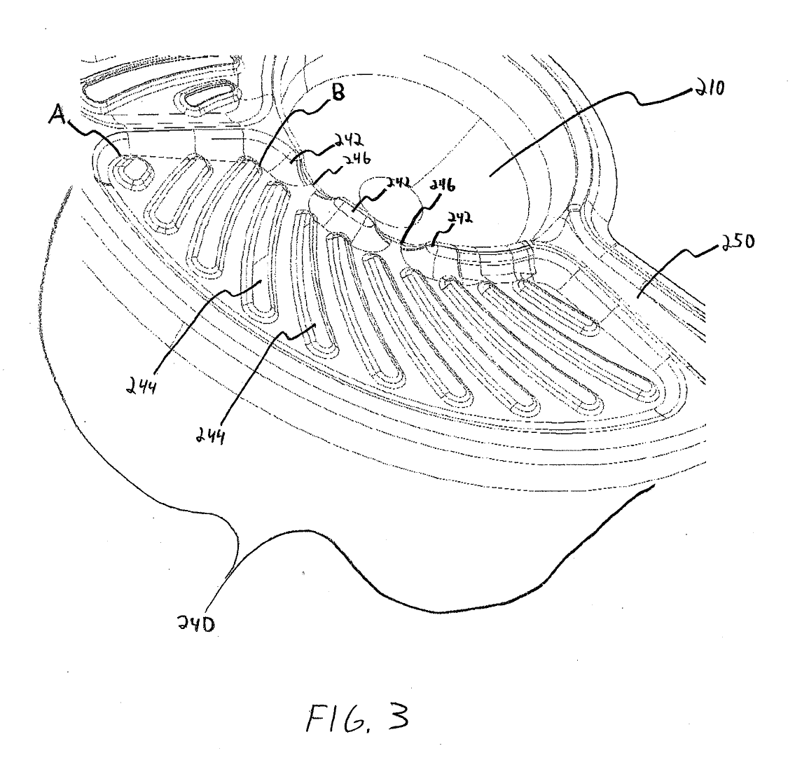

FIG. 3 illustrates an enlarged perspective view of the warming/draining section 240 of some embodiments. To facilitate draining back to reservoir 210, the warming/draining section 240 can be sloped downward from an outer section to an inner section. For example, the surface of the warming/draining section 240 can slope downwards from point A to point B. In some embodiments, the warming/draining section 240 can slope downwards at approximately a 5 degree angle. In other embodiments, a greater or smaller slope may be used. In preferred embodiments, the downward-sloping angle is enough to allow oil to flow into the reservoir 210 by means of gravity when the cooking platform 200 is in a horizontal position.

The warming/draining section 240 can include open slots 246 that allow oil, water, and other sauces to drain into the reservoir 210. While elevated grooves 244 can support food while oil, water, or other sauces drain back into the reservoir 210, blocking knobs 242 can act as a barrier to prevent food from falling into the reservoir 210. Thus, the warming/draining section 240 can provide the dual benefit of maintaining a supply of oil, water, and other sauces in the reservoir 210 while removing excess oil, water, and other sauces from foods.

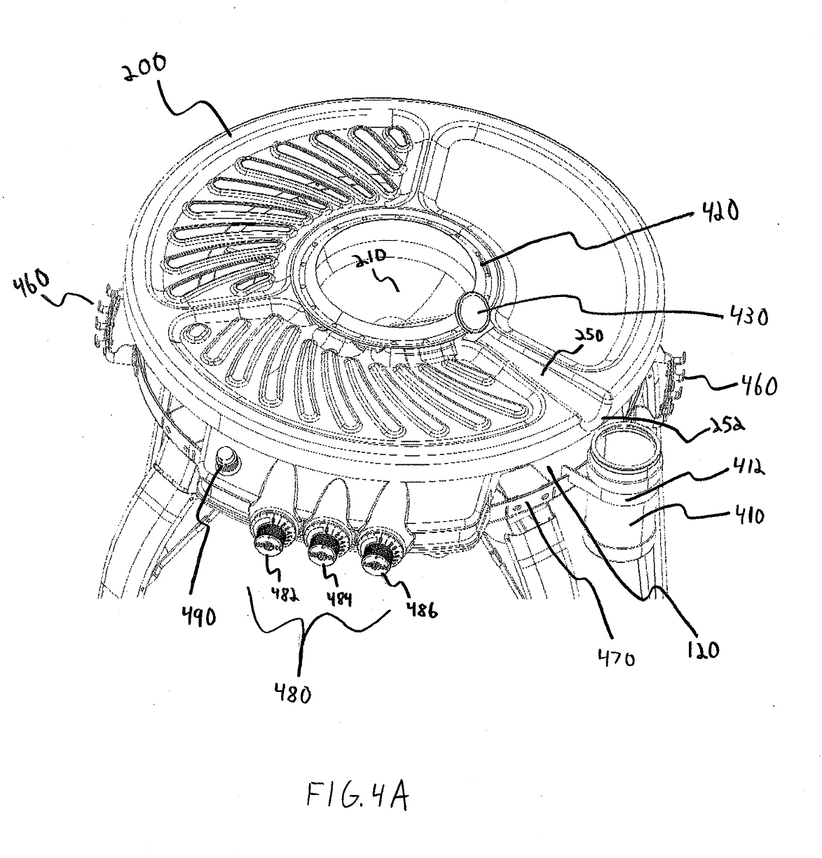

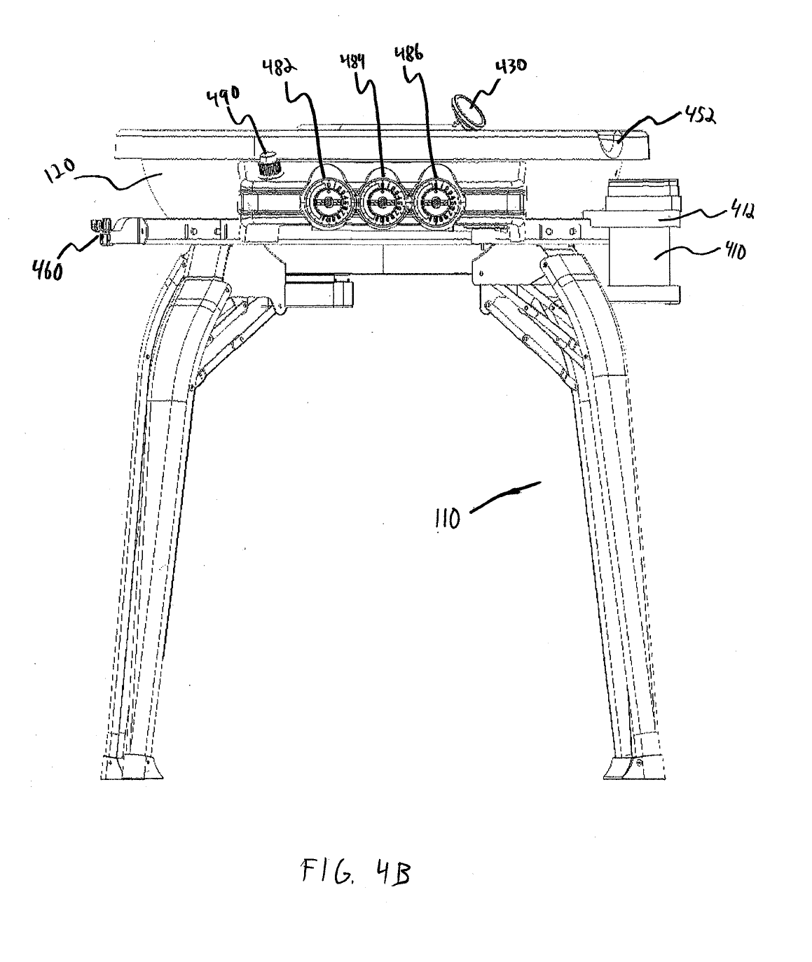

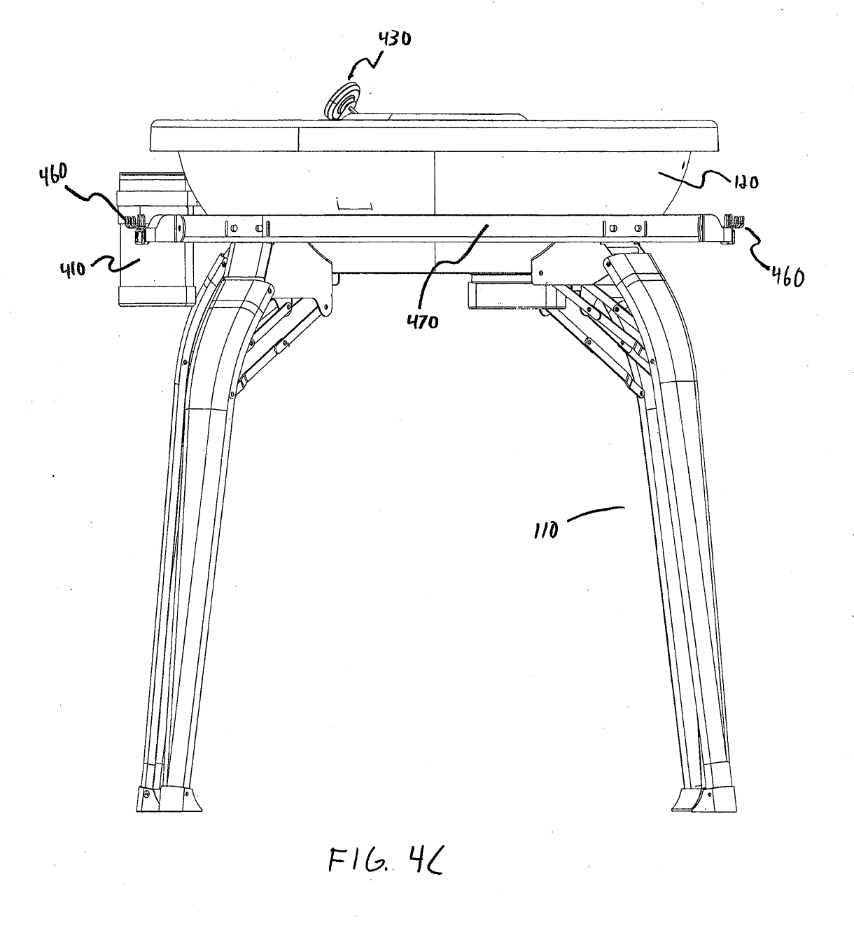

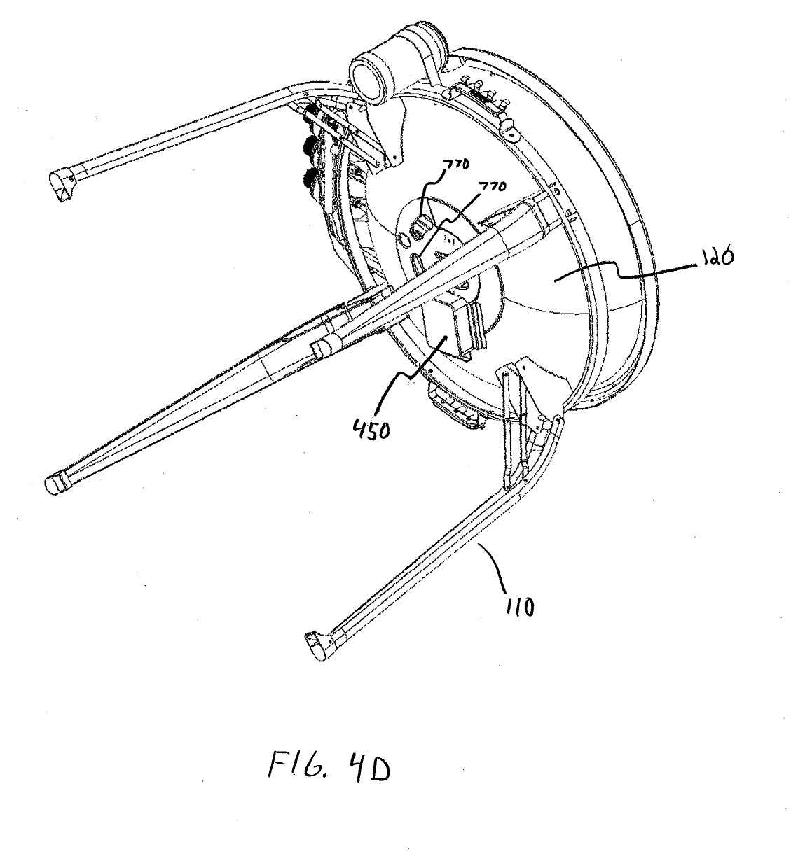

FIGS. 4A-4D illustrate a food preparation device of some embodiments. In particular, FIG. 4A illustrates a top perspective view of a food preparation device, FIG. 4B illustrates a front view a food preparation device, FIG. 4C illustrates a rear view of a food preparation device, and FIG. 4D illustrates a bottom perspective view of a food preparation device. The cooking platform 200 can rest on top of the inner housing 120. In addition, a frame ring rail 470 can surround the outer edge of the food preparation device. The frame ring rail 470 can be used to connect accessories to the food preparation device. For example, handles 460 can be attached to or integral with the frame ring rail 470 to ease transportation of the food preparation device. The handles 460 can include a silicone surface to facilitate gripping. In addition, side table 442 (shown in FIG. 1) can also connect to the frame ring rail 470.

A catch bottle 410 also can be attached to the frame ring rail 470. For example, a strap 412 can include notches at each end to interface with the frame ring rail 470. The strap 412 thereby allows the catch bottle 410 to be rigidly connected to the food preparation device. In alternative embodiments, the catch bottle 410 can be suspended by means of a wire hanger. In addition, any connection mechanism that allows the catch bottle 410 to remain in a vertical orientation while the cooking platform is moved may be used. Further, as shown in FIG. 4A, the catch bottle 410 preferably is affixed to the frame ring rail 470 such that it is in line with the drain chute 250 of the cooking platform.

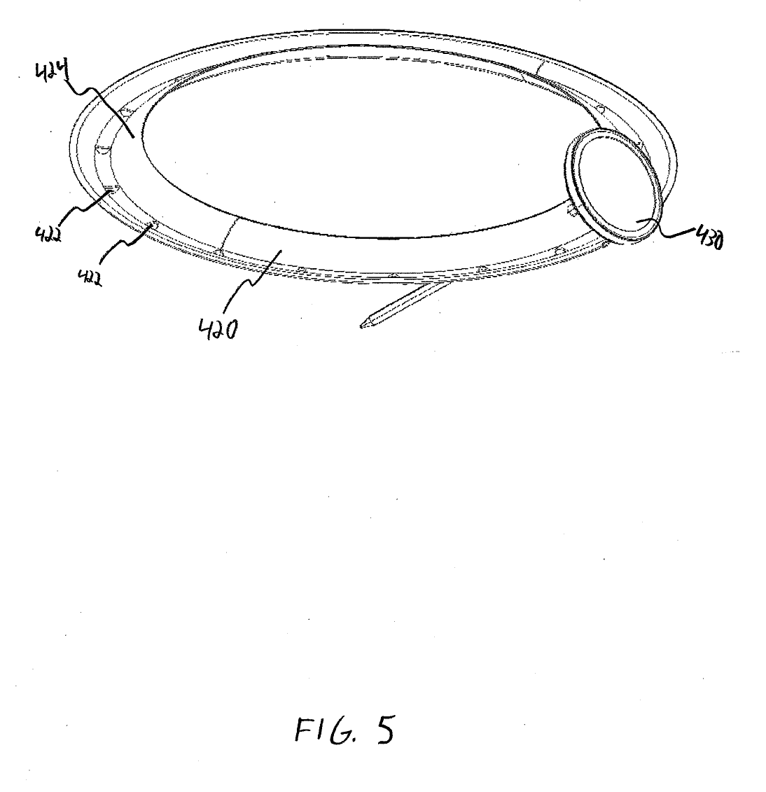

In some embodiments, a slosh ring 420 can be located on top of the cooking platform 200 surrounding the reservoir 210. The slosh ring 420 can serve several important functions. For example, the slosh ring 420 can provide a baffle function to prevent the liquid in the reservoir from sloshing out of the reservoir if the food preparation device is accidentally bumped or knocked. As shown in FIG. 5, which illustrates the slosh ring 420 in a perspective view, the slosh ring can include a flange 424. The flange 424 can be inwardly sloping to catch and prevent liquid from the reservoir from spilling over onto the other cooking sections of the cooking platform.

The slosh ring 420 can also include openings 422. If any liquid from the reservoir is accidentally spilled onto other sections of the cooking platform, the openings 422 can allow the spilled liquid to quickly drain back into the reservoir. Although the openings 422 shown in FIG. 5 encompass the entire perimeter of the slosh ring 420, in other embodiments any configuration of openings may be used to accomplish the intended purpose

The slosh ring 420 can also be adapted to receive a thermometer 430 for measuring the temperature of the liquid in the reservoir. When deep frying foods it is often advantageous to monitor the temperature of the oil used to fry foods. If the oil becomes too hot, the oil can burn. Optimal temperatures for frying can range from approximately 330 degrees to 400 degrees. As such, the thermometer 430 can include a color-coded portion to easily indicate to the operator of the food preparation device when the oil temperature is in the optimal range for frying. In addition, the slosh ring 420 can include a hole that holds the thermometer 430 in place for easy viewing and removing. This structure also allows the probe of the thermometer 430 to be placed in an optimal position for measuring the temperature of the liquid in the reservoir.

Turning back to FIGS. 4A and 4B, burner control knobs 480 can be located on the front of the food preparation device. In particular, infrared burner control knob 482 can be used to adjust the heat output of the infrared grill burner. Alternatively, in embodiments utilizing a standard grill burner, burner control knob 482 can be used to adjust the heat output of the grill burner. In addition, fryer/steamer burner control knob 484 can be used to adjust the heat output of the fryer/steamer burner, and griddle control knob 486 can be used to adjust the heat output of the griddle burner. Burner control knobs 482, 484, and 486 can be operated to independently control the heat output of the grill burner, fryer/steamer burner, and griddle burner. In alternative embodiments, a single burner control knob can be used.

An igniter 490 can be located on the front of the food preparation device. The igniter 490 can be used to ignite the fryer/steamer burner, the grill burner, the griddle burner, and any other burners that can be included. In preferred embodiments, the food preparation device comprises a fryer/steamer burner, a grill burner, and a griddle burner. In alternative embodiments, the food preparation device can comprise fewer or additional burners. For example, as additional cooking surfaces are added to the cooking platform, corresponding burners can be added to the food preparation device. In some alternative embodiments, the cooking device comprises four, five, six, or more cooking surfaces. In those embodiments, additional burners and controls can be added to provide heating for each cooking surface. It addition, any cooking surface or combination of cooking surfaces can share one or more burners. In the embodiment illustrated in FIG. 4A, the igniter 490 is located next to the burner controls and extends in a vertical direction. However, in preferred embodiments, the igniter 490 is located next to the burner controls and extends in a horizontal direction, as shown in FIG. 1. In other embodiments, burner controls can be placed in other locations and could be separated.

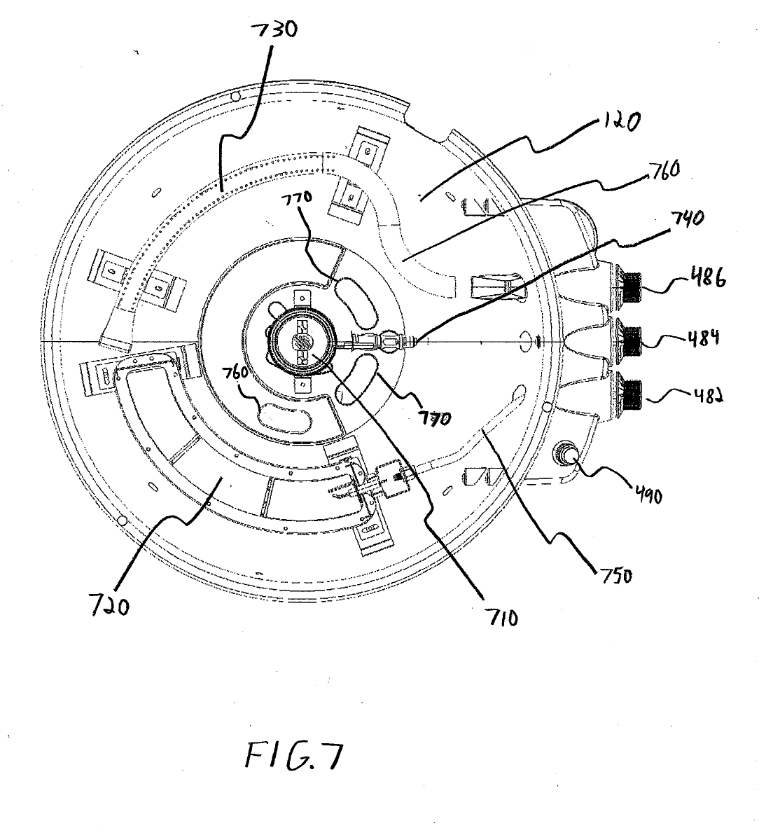

As shown in FIG. 4D, a grease pan 450 can attach to the underside of the lower housing 120. The grease pan 450 can be used to collect any grease that drips from the cooking platform 200 to the lower housing 120. The grease pan 450 can be useful when grilling foods, as grease often can drip through the open slots located in the grilling section of the cooking platform. As described in more detail with respect to FIG. 7, vent openings 770 in the lower housing 120 can include upward oriented lips or walls that prevent grease from dripping through vent openings 770. In contrast, the vent opening in line with the grease pan (not shown in FIG. 4D) can include a downward oriented lip, or no lip at all, so that grease may freely pass into the grease pan 450.

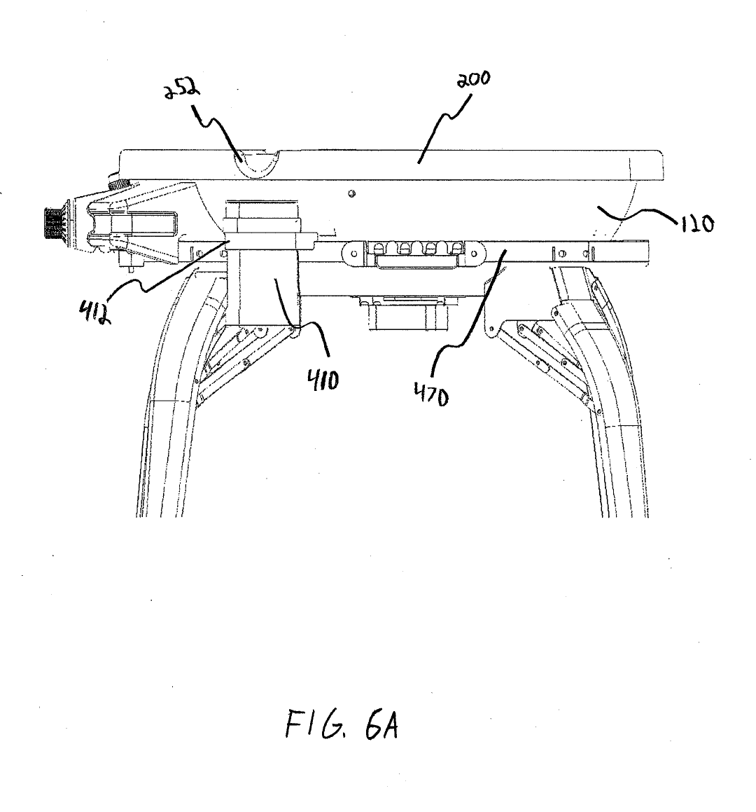

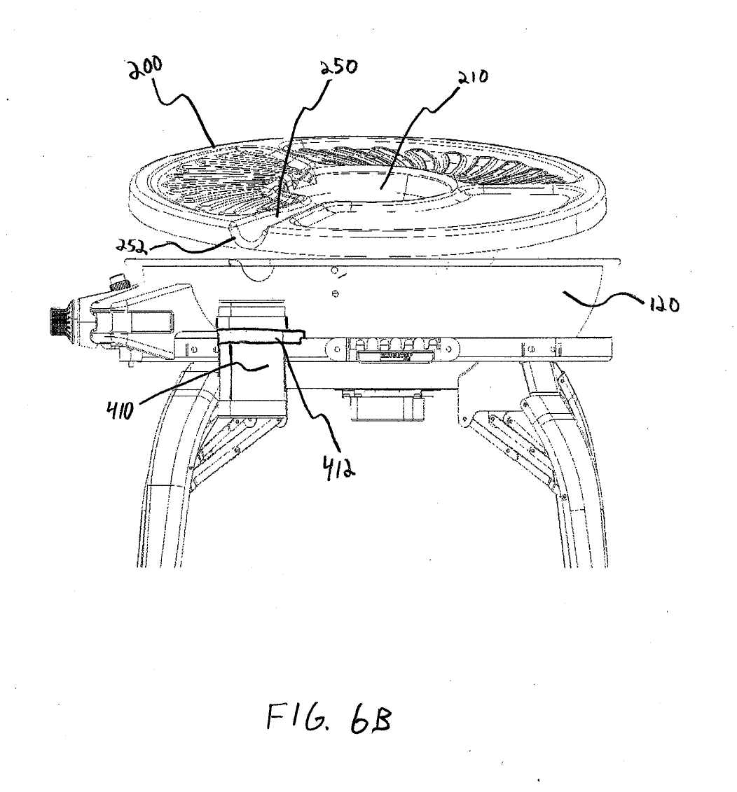

FIG. 6A is a partial side view of a food preparation device of some embodiments showing the cooking platform 200 in a horizontal position. The mouth 252 of the drain chute 250 can be aligned with the catch bottle 410. As shown in FIG. 6B, the cooking platform 200 can be lifted and tipped at an angle to cause the liquid or viscous food contents of the deep frying/boiling/steaming/stir frying reservoir 210 to drain down the drain chute 250 and into the catch bottle 410 for easy cleanup and/or disposal. In addition, in embodiments in which the catch bottle 410 is suspended by means of a wire hanger, the contents of the reservoir 210 may be drained into the catch bottle 410 by tilting the entire food preparation device.

FIG. 7 is a top view of a food preparation device of some embodiments with the cooking platform removed to illustrate the heating elements. As shown, the deep frying/boiling/steaming/stir frying burner 710 can be round in shape. Alternatively, the deep frying/boiling/steaming/stir frying burner 710 can be any shape or size that satisfies the heating requirements of the liquid used to fry or boil foods in the reservoir 210. The deep frying/boiling/steaming/stir frying burner 710 can have an independent control knob 484 and an independent fuel delivery train 740. In alternative embodiments, other heating elements may be used. For example, the structure of the inner housing can be adapted to use charcoal as the fuel source to provide heat to each cooking section of the food preparation device. In other embodiments, additional heating elements for additional cooking sections may be included.

Grill burner 720 can be used to heat the grilling section 220 of the cooking platform. The grill burner 720 can vary in size to correspond to the size of the grilling section 220. In preferred embodiments, the grill burner 720 comprises an infrared grill burner. The infrared grill burner can have a curved shape such that it corresponds to the shape of the grilling section of the cooking platform. The curved shape of the infrared grill burner that matches the shape of the grilling surface allows the infrared grill burner to provide a more even distribution of heat to the grilling surface. The infrared grill burner can provide in excess of 650° F. temperatures, allowing foods to be cooked in less time than would be required by a conventional grill burner. In alternative embodiments, the grill burner 720 can include a conventional grill burner. As shown, the grill burner 720 can have an independent control knob 482 and an independent fuel delivery train 750.

In addition, the infrared grill burner can include one or more ceramic plates. The ceramic plates generally include numerous ports that are arranged on the surface of the ceramic plates. The infrared grill burner generally radiates heat as gas exiting the ports is ignited, burns, and heats, the ceramic plates. The infrared grill burner can be configured to display a logo, design, pattern, text, or other visual indicia that glows when the ceramic plates are heated above a certain temperature. To form the logo, design, pattern, text, or other visual indicia, the size of the ports on the surface of the grill burner can be varied. Also, to form the logo, design, pattern, text, or other visual indicia, the height or thickness of the ceramic surface can be varied.

The griddle burner 730 can be used to heat the griddle/frying/cooking section 230 of the cooking platform. The griddle burner 730 can vary in size to correspond to the size of the griddle/frying/cooking section 230. As shown, the griddle burner 730 can have an independent control knob 486 and an independent fuel delivery train. In alternative embodiments, the deep frying/boiling/steaming/stir frying burner 710, grill burner 720, and the griddle burner 730 can be controlled by a single burner control knob, which simplifies the fuel delivery system 760.

The lower housing 120 can have an angle that directs grease drippings to the grease pan 450. In addition, the lower housing 120 can include opening 760 and openings 770 that provide ventilation. Opening 760 can include a downward oriented lip that allows grease to drip into the grease pan 450. In preferred embodiments, grease pan 450 can be located under opening 760 to catch any dripping grease. In contrast, openings 770 can include upward oriented lips or walls that blocks grease from dripping out.

The structure of the lower housing 120 can protect the burners and fuel delivery systems while securing the control knobs, a regulator, and a valve outside the housing. The protection provided by the lower housing 120 can be advantageous when the food preparation device is in storage and/or transport.

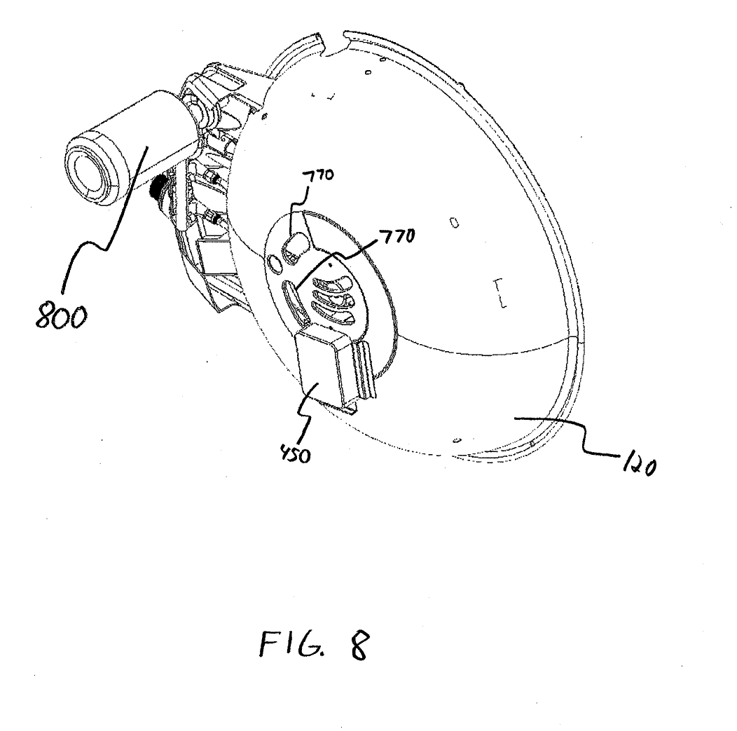

FIG. 8 illustrates a bottom perspective view of selected portions of the food preparation device that shows connection of a fuel canister 800. In preferred embodiments, the fuel canister 800 can be a standard one-pound canister of propane. Alternatively, the food preparation device can accommodate a 20 pound canister of propane that can be connected to a fuel delivery system via a hose. In addition, other embodiments may utilize other canisters of varying size and shape. Furthermore, any other suitable fuel source, including liquid petroleum gas, natural gas, charcoal, gelled petroleum, electricity, green gas, or any other fuel also can be used. Furthermore, any combination of liquid petroleum gas, propane, natural gas, charcoal, gelled petroleum, electricity, and green gas can be used to provide energy for the heating elements.

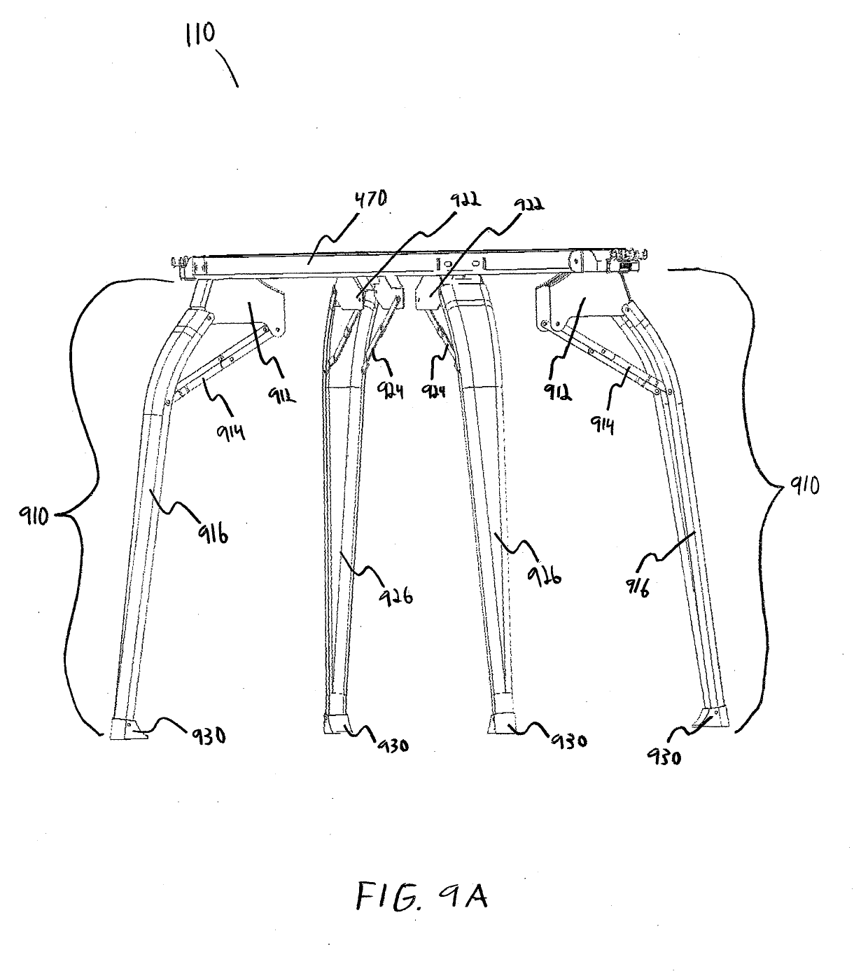

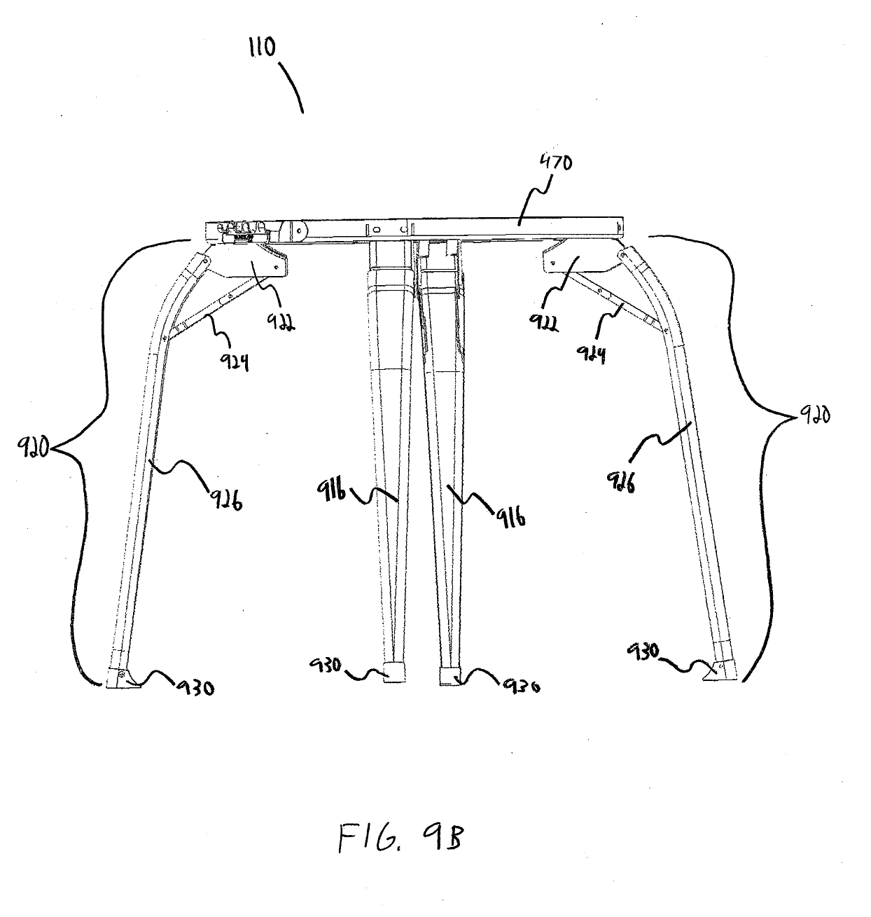

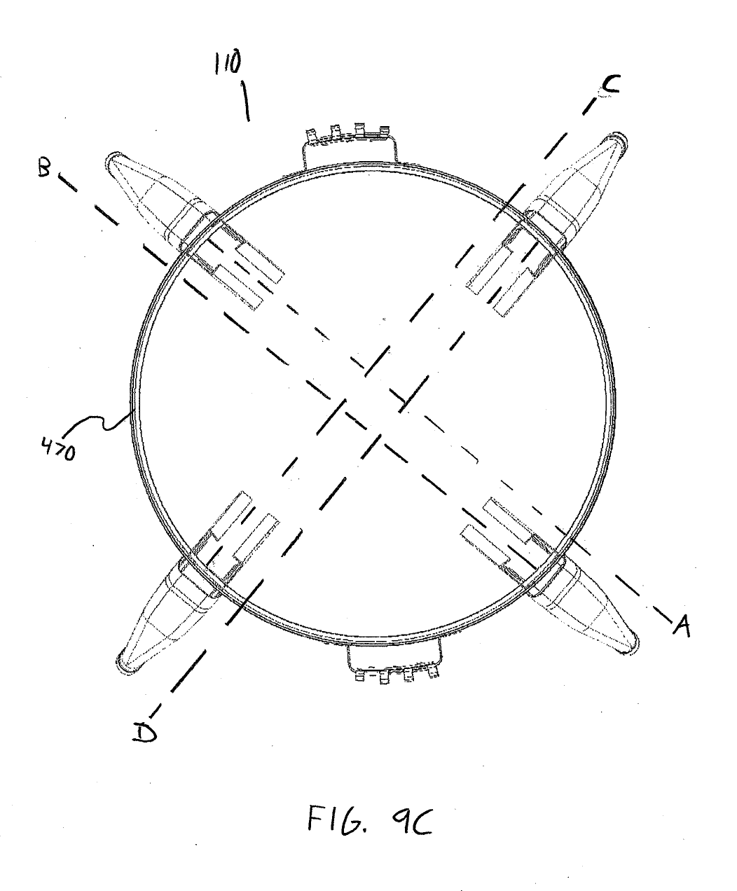

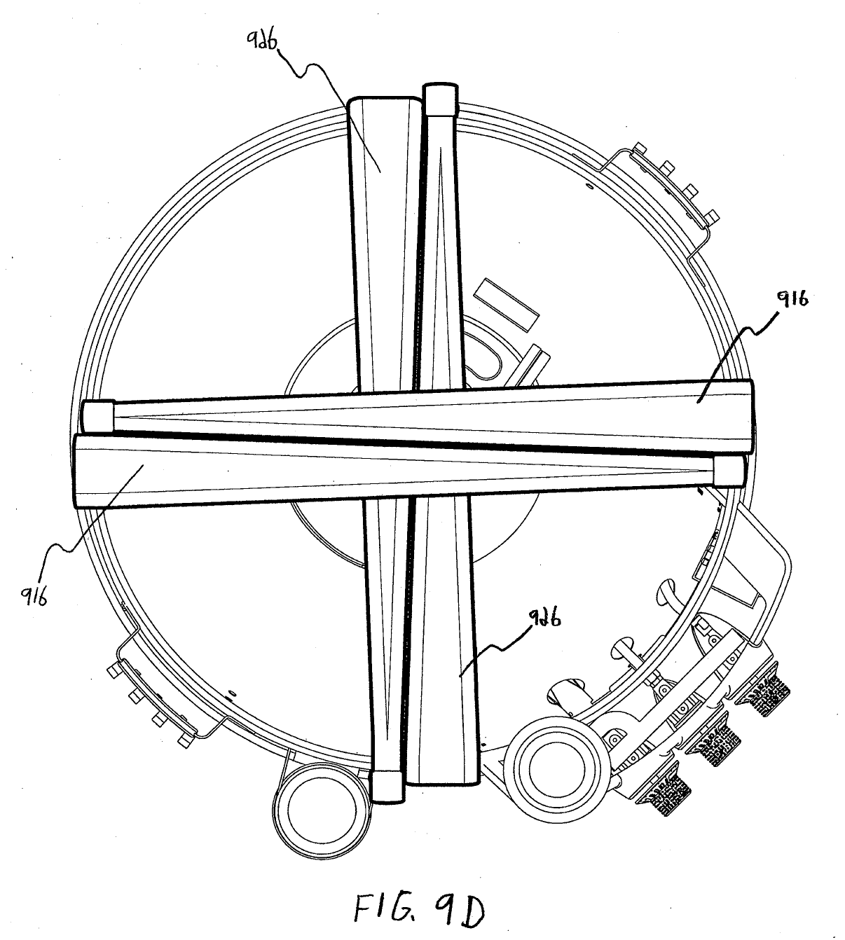

FIGS. 9A-9D illustrate the legs of some embodiments of a food preparation device in varying positions. The collapsible stand 110 of the food preparation device generally comprises a frame ring rail 470 and four leg assemblies. A first pair of leg assemblies 910, shown in FIG. 9A, can include large mounting brackets 912, support bars 914, short legs 916, and rubber feet 930. A second pair of leg assemblies 920, shown in FIG. 9B, can include small mounting brackets 922, support bars 924, long legs 926, and rubber feet 930. The first pair of leg assemblies 910 and second pair of leg assemblies 920 can be constructed of steel. Large mounting brackets 912 and small mounting brackets 922 can facilitate attachment of the first pair of leg assemblies 910 and the second pair of leg assemblies 920, respectively, to the frame ring rail 470. The large mounting brackets 912 and small mounting brackets 922 can also be connected to the inner housing 120 of a food preparation device. Working together, the first pair of leg assemblies 910 and second pair of leg assemblies 920 provide the stand 110 with needed stability. For example, a stable stand reduces the risk that the food preparation device could be accidentally knocked over. In addition, rubber feet 930 also provide added stability.

As shown in FIG. 9B, the pair of short legs 916 can be slightly horizontally displaced with respect to each other. Similarly, the pair of long legs 926 can be slightly horizontally displaced with respect to each other. FIG. 9C, which provides a top view of the stand 110, shows the slight horizontal displacement of each pair of legs. Dotted lines A, B, C, and D run through the center axis of each leg. The horizontal displacement of each pair of legs facilitates easy and compact folding. For example, horizontal displacement allows each pair of legs to be folded side by side, as shown in FIG. 9D.

The relative size difference between small mounting brackets 922 and large mounting brackets 912 can allow the pair of short legs 916 to be folded on top of the pair of long legs 926, further facilitating compact folding for ease of transport. In particular, the small mounting brackets 922 can have less height than the large mounting brackets 912, whereas the long legs 926 can have greater height than the short legs 916. When considered together, the combined height of the long legs 926 and small mounting brackets 922 is approximately the same as the combined height of the short legs 916 and large mounting brackets 912. In preferred embodiments, the legs can vary in height from approximately 29 to 31 inches. In other embodiments, the legs can be less or greater in height.

A method of collapsing the stand can include first folding the pair of long legs 926 into a collapsed position. Next, the pair of short legs 916 can be folded into a collapsed position. Because the pair of short legs 916 are attached to the large mounting bracket 912, the short legs 916 can fold directly over the pair of long legs 926. FIG. 9D illustrates the stand of the food preparation device in a collapsed position. After the stand is collapsed, the food preparation device fits into a carrying bag (not shown) for ease transport. In addition, in preferred embodiments, the food preparation device weighs less than 40 lbs., further adding to ease of transport.

Preferred embodiments utilize the folding leg structure described above with reference to FIGS. 9A-9D. However, in alternative embodiments, other leg structures may be utilized. For example, a stand comprising three legs or more than four legs can be used with a food preparation device. In addition, stands that include legs with traditional folding structures can also be used. In other alternative embodiments, the food preparation device may be constructed without legs or with short legs. Such embodiments may be advantageous in table-top versions of the food preparation device or in versions in which the food preparation device is permanently installed.

Several methods of operating the food preparation device disclosed herein will now be described. In one method, a user can operate the heating element of any cooking section while the heating element for other cooking sections remains off. For example, a user may ignite the grill burner 720 and use the grilling section 220 of the cooking platform to grill while the deep frying/steaming/stir frying reservoir 210, griddle/cooking/frying section 230, and warming/draining section 240 are unused. While grilling, the user may operate the grill burner control knob 484 to control the heat output of the grill. Similarly, the user may cook using the deep frying/steaming/stir frying reservoir 210 or griddle/cooking/frying section 230 while the other cooking sections are unused. Furthermore, in some methods, the warming/draining section 240 can be by itself at a particular time to warm food without cooking food on the other cooking sections.

In another method, a user can simultaneously operate any combination of two cooking surfaces. For example, a user can use the grilling section 220 to cook burgers while simultaneously using the deep frying/steaming/stir frying reservoir 210 to make french fries. Similarly, a user can simultaneously use the griddle/cooking/frying section 230 and the warming/draining section 240. For example, a user can the griddle/cooking/frying section 230 to toast buns and the warming/draining section 240 to keep buns warm after being toasted.

In another method, a user can simultaneously operate any combination of three cooking surfaces. For example, a user can use the grilling section 220 to cook burgers while simultaneously using the deep frying/steaming/stir frying reservoir 210 to make french fries and the griddle/cooking/frying section 230 to toast buns.

In another method, a user can simultaneously operate any combination of four surfaces. For example, a user can use the grilling section 220 to cook burgers while simultaneously using the deep frying/steaming/stir frying reservoir 210 to make french fries, the griddle/cooking/frying section 230 to toast buns, and the warming/draining section 240 to keep burgers, fries, and buns warm.

In another method, a user can vary the timing at which the user begins using each cooking surface. For example, the user may wish to have several food items be ready at the same time, while certain food items may take longer to cook and prepare than others. In a particular example, it may require 5 minutes to prepare food A using the frying/steaming/stir frying reservoir but only 2 minutes to grill food B using the grilling section and infrared grill burner. So, a user may begin cooking food A using the frying/steaming/stir frying reservoir and after 3 minutes begin cooking food Busing the grilling section, such that the meal of foods A and B are ready for consumption at the same time, five minutes after the user began the cooking process. While this example has been described in reference to foods A and B, it can be appreciated that with variations in foods and combination of cooking surfaces being used, the time to begin cooking any particular food item can be varied accordingly.

It can be appreciated that many of the specific embodiments of the food preparation device described above and illustrated in the drawings include several unique and ornamental qualities arranged in a particular way. These ornamental qualities are non-functional. The food preparation device can be designed in a number of different ways while staying within the scope of the present disclosure.

The features and attributes of the specific embodiments disclosed above may be combined in different ways to form additional embodiments, all of which fall within the scope of the present disclosure. Although this invention has been described in terms of certain preferred embodiments, other embodiments that are apparent to those of ordinary skill in the art, including embodiments which do not provide all of the benefits and features set forth herein, are also within the scope of this invention. Accordingly, the scope of the present invention is defined only by reference to the appended claims.

What is claimed is:

1. A multi-functional platform food preparation device comprising:
a cooking platform comprising:
a first section that includes a reservoir for deep frying, steaming, stir frying, and boiling;
a second section for grilling;
a third section for cooking and frying by griddle; and
a fourth section for warming and draining.

2. The food preparation device of claim 1, wherein the cooking platform is formed from a unitary piece of metal.

3. The food preparation device of claim 1, wherein the cooking platform further comprises a drain chute that connects the reservoir with an outer edge of the cooking platform.

4. The food preparation device of claim 3, further comprising a catch bottle, and wherein the drain chute can be aligned with the catch bottle to facilitate disposal of liquid from the reservoir.

5. The food preparation device of claim 4, wherein the catch bottle is rigidly attached to the food preparation device.

6. The food preparation device of claim 1, wherein the cooking platform is formed in a circular or disk shape, wherein the reservoir is located in the center of the cooking platform, and wherein the second, third, and fourth sections are adjacent to each other and surround the reservoir.

7. The food preparation device of claim 1, wherein the fourth section comprises slots that allow liquids to drain into the reservoir and blocking knobs that prevent prepared food from sliding into the reservoir.

8. The food preparation device of claim 1, further comprising a slosh ring positioned around an upper edge of the reservoir, wherein the slosh ring comprises a flange that aids in preventing a liquid in the reservoir from spilling.

9. The food preparation device of claim 8, wherein the slosh ring further comprises openings that allow liquid that has spilled out of the reservoir to drain back in.

10. The food preparation device of claim 8, further comprising a thermometer, wherein the slosh ring is configured to receive the thermometer.

11. The food preparation device of claim 1, further comprising:
a first burner for heating the reservoir for deep frying, steaming, stir frying, and boiling;
a second burner for heating the second section for grilling; and
a third burner for heating the third section for cooking and frying by griddle.

12. The food preparation device of claim 11, wherein the second burner is an infrared grill burner.

13. A method of cooking comprising:
deep frying, steaming, stir frying, or boiling a first food;
grilling a second food;
cooking or frying by griddle a third food; and
warming or draining a fourth food;
wherein the deep frying, steaming, stir frying, or boiling of a first food, grilling of a second food; cooking or frying by griddle of a third food; and warming or draining of a fourth food occurs on a single cooking platform.

14. The method of cooking of claim 13, further comprising, after cooking has completed and the cooking platform has cooled, using a drain chute in the cooking platform to dispose into a catch bottle the liquid used to deep fry, steam, stir fry, or boil the first food.

15. The method of cooking of claim 13, further comprising checking the temperature of a thermometer located in the liquid being used to deep fry, steam, stir fry, or boil the first food.

16. The method of claim 13, wherein an infrared grill burner is used to grill the second food.

17. A multi-functional platform food preparation device comprising:
a first pair of leg assemblies, wherein the first pair of leg assemblies includes a first pair of mounting brackets and a first pair of legs;
a second pair of leg assemblies, wherein the second pair of leg assemblies includes a second pair of mounting brackets and a second pair of legs;
wherein the first pair of mounting brackets are larger than the second pair of mounting brackets; and
wherein the second pair of legs are longer than the first pair of legs.

18. The food preparation device of claim 17, wherein the legs of the first pair of leg assemblies are displaced with respect to each other to enable side by side folding; and wherein the legs of the second pair of leg assemblies are displaced with respect to each other to enable side by side folding.

19. The food preparation device of claim 18, further comprising a carrying bag, wherein the food preparation device fits into the carrying bag when the first and second pair of leg assemblies are in a folded state.

20. The food preparation device of claim 17, further comprising a cooking platform, wherein the cooking platform includes a first section for deep frying, steaming, stir frying, and boiling, a second section for grilling, a third section for cooking and frying by griddle, and a fourth section for warming and draining.
